(19) United States
(12) Patent Application Publication
(10) Pub. No.: US 2018/0221683 A1
(43) Pub. Date: Aug. 9, 2018

(54) Portable Phototherapy Device for Treatment of Seasonal Affect Disorder Symptoms

(71) Applicants: Kevin Kang, Erie, PA (US); Sophia Diserio, New York, NY (US); Braden Pellowski, Austin, TX (US)

(72) Inventors: Kevin Kang, Erie, PA (US); Sophia Diserio, New York, NY (US); Braden Pellowski, Austin, TX (US)

(21) Appl. No.: 15/806,155

(22) Filed: Nov. 7, 2017

Related U.S. Application Data
(60) Provisional application No. 62/455,551, filed on Feb. 6, 2017.

(51) Int. Cl. A61N 5/06; H05B 33/08; F21V 3/00; F21V 23/02; F21V 21/088

(52) U.S. Cl. CPC A61N 5/0618; H05B 33/0845; F21Y 2115/10; F21V 23/02; F21V 21/088; F21V 3/00

(57) ABSTRACT

A phototherapy device for treatment of seasonal affective disorder that comprises a housing that further comprises a light source, a mounting device that allows the phototherapy device to be mounted to any object or surface to allow the light source to be directed at a user's eye, intensity control for modulating the intensity of light emitted by the light source, and a power source to supply power to the phototherapy device. The housing has a concave surface onto which the light source is mounted. The concave surface imparts a degree of curvature to the light source to enhance the amount of light directed towards the eye of a user adjusted for the typical distance of the light source from the user.

### BACKGROUND

In many parts of the world, winter months are accompanied by shorter days, overcast skies, freezing temperatures, and heavy snow. These elements work in unison to keep people indoors and, consequently, out of the sun. Exposure to sunlight plays a critical role in regulating the brain's hormones. As such, when an individual spends very little time outdoors for months on end, he or she is more prone to moodiness, anxiety, fatigue, and overall sadness. SAD, or Seasonal Affective Disorder, is a condition that is ascribed to individuals who experience such symptoms. Whether diagnosed or not, SAD affects millions of people across the globe on a daily basis. In the northern United States, nearly 10% of people experience symptoms of SAD. Women, people who live at a higher latitudes, and people between the ages of 15 and 55 are at greater risk of developing SAD. College students are particularly prone to SAD symptoms, as they must cope with winter weather as well as the added stress of their academics. Moreover, U.S. community surveys indicate that the incidence of SAD is highest in U.S. states at high latitudes; the disorder is most prevalent in states like New Hampshire and least prevalent in states like Florida.

The leading remedy for SAD is light therapy. Seasonally affected individuals can purchase lights that simulate sunlight, thus balancing hormone and neurotransmitter levels in the brain. These lights, called “happy lamps,” while effective, are not convenient to use. They are cumbersome, generally aesthetically displeasing, and the vast majority of existing models require outlet power. For these reasons, happy lamps are inconvenient to transport and use frequently. The most important issues with contemporary devices include portability, aesthetics, cost, and limited options for power supply and light intensity. A smaller, more portable happy lamp would be an improvement upon the happy lamps used today. Summarily, although phototherapy is a clinically-proven method for effectively reducing SAD symptoms, there are no phototherapy devices that allow users to conveniently use their lamps throughout the day. What is presented is a more convenient happy lamp that is portable for treatment of SAD symptoms that is specifically designed to address the indicated deficiencies.

### SUMMARY

What is presented is a phototherapy device for treatment of seasonal affective disorder that comprises a housing that further comprises a light source, a mounting device that allows the phototherapy device to be mounted to any object or surface to allow the light source to be directed at a user's eye, intensity control for modulating the intensity of light emitted by the light source, and a power source to supply power to the phototherapy device. The housing has a concave surface onto which the light source is mounted. The concave surface imparts a degree of curvature to the light source to enhance the amount of light directed towards the eye of a user adjusted for the typical distance of the light source from the user.

The phototherapy device may include a lens positioned to obscure the individual elements of the light source and to diffuse the light from the light source while maintaining the brightness of the light source. The mounting device allows the phototherapy device to be mounted to a laptop computer, a desktop monitor, a television screen, a handheld electronic device, a clipboard, a table, etc. The mounting device may be a clamp, an adhesive, a screw, an elastic band, a tripod, hook and loop fastening strips, a stand, etc. The power source may be a USB connection, a micro-USB connection, batteries, a wired connection to a power outlet, etc. The intensity control is any one of a potentiometer or a power modulator. The light source provides light in any of full spectrum visible spectrum, polychromatic white light, blue light, or a tunable spectrum and is any one of LED lights, incandescent bulbs, fluorescent bulbs, or compact fluorescent bulbs.

Those skilled in the art will realize that this invention is capable of embodiments that are different from those shown and that details of the apparatus and methods can be changed in various manners without departing from the scope of this invention. Accordingly, the drawings and descriptions are to be regarded as including such equivalent embodiments as do not depart from the spirit and scope of this invention.

### BRIEF DESCRIPTION OF DRAWINGS

For a more complete understanding and appreciation of this invention, and its many advantages, reference will be made to the following detailed description taken in conjunction with the accompanying drawings.

FIG. 1 depicts an embodiment of the phototherapy device mounted to the screen of a laptop computer; and

FIG. 2 depicts the phototherapy device of FIG. 1 with a cutout of the lens to show the concave surface onto which the LED lights of the light source are mounted.

### DETAILED DESCRIPTION

Referring to the drawings, variations of corresponding parts in form or function that are depicted in the figures are described. It will be understood that variations in the embodiments can generally be interchanged without deviating from the invention.

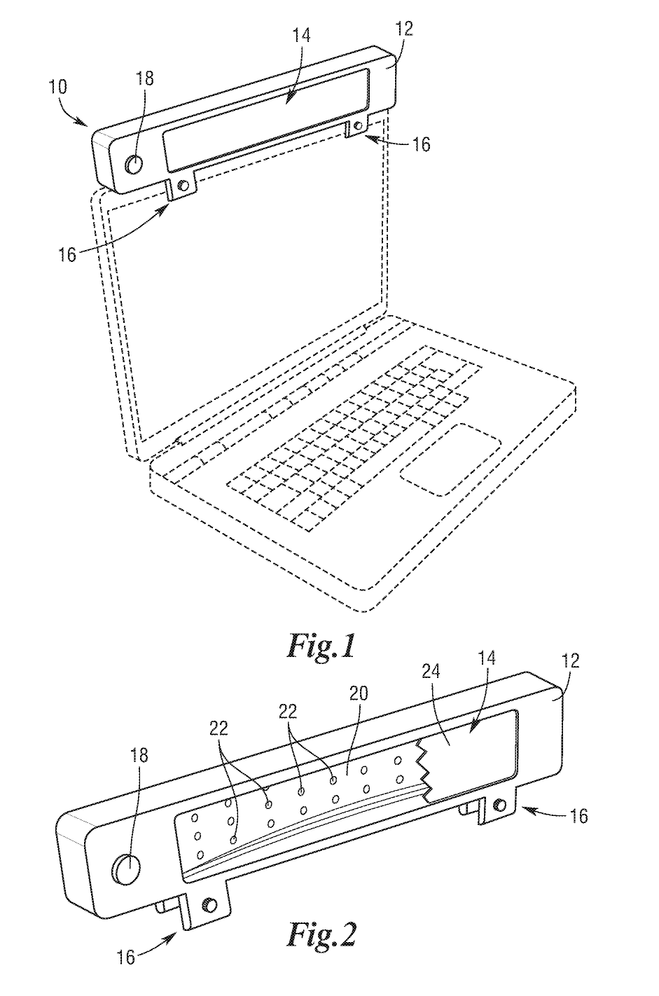

The portable phototherapy device for treatment of seasonal affective disorder presented is portable and discrete enough to be used in public workspaces. FIG. 1 shows the portable phototherapy device 10 mounted to the display of a laptop computer but it is understood that it may be mounted to any device that can receive it such as a desktop monitor, a television screen, a handheld electronic device, a clipboard, a table, or any other device.

Referring to FIGS. 1 and 2, the phototherapy device 10 comprises a housing 12 that further comprises a light source 14 and a mounting device 16 that allows the phototherapy device to be mounted to any object or surface to allow the light source 14 to be directed at a user's eye. An intensity control 18 modulates the intensity of the light emitting from the light source 14. A power source (not shown) is required to supply power to the phototherapy device 10. The housing 12 has a concave surface 20 onto which the light source 14 is mounted. The concave surface 20 has a degree of curvature designed to enhance the amount of light reaching the eye of a user adjusted for the typical distance of the light source 14 from the user. The light source 14 in the embodiment shown in the figures comprises a series of LED lights 22 arranged to follow along the concave surface 20 of the housing and a lens 24 positioned to obscure the individual elements of the LED lights 22 and to diffuse the light from the light source 14 while maintaining the brightness of the light source 14.

The mounting device 16 in the embodiment shown in the figures are clamps made of two legs and a nylon screw. This configuration allows the phototherapy device 10 to be placed on the screen of any computer as long as it is less than the distance between the legs. The embodiment shown uses nylon screws which assists in securing the phototherapy device 10 at a reduced risk of hurting the surface underneath as nylon plastic is fairly soft compared to the metal of regular screws. The legs of the mounting device 16 in this embodiment are sized so that the phototherapy device 10 would rest right on the top of screens without covering cameras or other accessories that may be located below the phototherapy device 10. It will be understood that the width of the legs may be varied and that the screw may be other than nylon and may also be any other device like clips, buttons, or other fasteners to secure the phototherapy device 10 to whatever it is mounted to. The mounting device 16 may be a clamp, an adhesive, a screw, an elastic band, a tripod, hook and loop fastening strips, a stand, or other devices that allow the phototherapy device 10 to mount in a way that the light source 14 may be properly directed to a user.

The concave surface 20 is curved so that the LED lights 22 mounted against the surface is correspondingly curved to direct the outputted light towards a user's eyes and reduce the amount of light that is lost to the environment. While the embodiment shown in the figures shows a straight lens 24, it will be understood that the lens may also be curved to match the concave surface 20. The concave surface 20 imparts a degree of curvature to the light source 14 to enhance the amount of light directed towards the eye of a user adjusted for the typical distance of the light source 14 from the user.

The intensity control 18 is a potentiometer or a power modulator that modulates the intensity of light emitted by the light source 14. The intensity control 18 is a knob in the embodiment shown in the figures but it could be any other device such as a touch sensor.

The light source 14 shown in the figures comprises a strip of LED lights 22 as the source of the therapeutic light. The exact type of LED circuit and light can be varied in different embodiments, including individual diffused round bulbs, individual flat bulbs, and strips of flat bulbs, all varying in brightness, forward voltage, and forward current. The LED lights could be LED lights, incandescent bulbs, fluorescent bulbs, compact fluorescent bulbs, or any other light emitting device. The preferred embodiment uses white LEDs because full-spectrum white light is the clinical standard for phototherapy. However, the specific light frequency could be varied or targeted as required for a particular treatment regime. The light source 14 could provide light in any of full spectrum visible spectrum, polychromatic white light, blue light, or a tunable spectrum.

The primary purpose of the lens 24 is to diffuse the LED lights 22 so that the individual LEDs and circuitry are obscured while the maintaining brightness of the light source 14. It would be understood that the lens 24 is not required for therapy and some embodiments may not include a lens 24

The phototherapy device 10 also includes a power source (not shown) to power the light source 14. The power source could be any that would provide sufficient power to the phototherapy device 10. It could be, for example, a standard power cord, a retractable power cord, removable batteries, a rechargeable battery pack, etc. If it is any version of a power cord, it could be a standard plug to power the phototherapy device 10 from a standard wall power outlet, or it could be USB or micro-USB connection to connect the phototherapy device 10 to the device onto which it is mounted. If it is a USB plug, it would make the phototherapy device 10 truly portable as it could be powered by the laptop computer or device onto which it is mounted.

The light intensity from the light source 14 was quantified at various angles from the phototherapy device 10, light intensity at various distances from the phototherapy device 10, spectral profiles of various embodiments, and battery life consumption of various embodiments. The benchmark testing consisted of three instrument tests as well as user testing on each embodiment. First, a light meter was used to measure lux or luminous flux into the eye. Two tests were conducted, one measuring light intensity as it varies with the angle relative to the user, the other light intensity as it varies with distance. The light meter sensor was held up to a user's right and left eyes and moved the phototherapy device 10 around the user, measuring the lux at directly in front of the user's eyes, ₄₅ degrees to the right and left of the user, and 90 degrees to the right and left of the user. This measured the amount of light reaching the eye as the phototherapy device 10 was moved around the user. These measurements were made on the dimmest, middle, and brightest settings, knowing that the brightest setting was uncomfortably bright and impractical for the user.

Using the light meter again, the phototherapy device 10 was positioned in front of the user at 40 cm, which is the average measured distance from a users' eyes to the top of their laptop screen. The light intensity was measured at different distances ranging from 0 cm away from the phototherapy device 10 to 50 cm away. The purpose of this testing was to determine the optimal distance at which a user could be sitting and still feel effects of the light without straining their typing posture. Both curved and straight light sources were measured to determine if directing the light to a focus increased intensity and decreased the amount of light lost. Next, a spectrometer was used to take spectral readings and determine the spectral profile of the phototherapy device 10. Although each phototherapy device 10 used full spectrum bulbs, different embodiments had different peak wavelengths. Finally, user testing was conducted with various phototherapy device 10 embodiments. The user testing format consisted of 10 to 14 users who sat in front of each phototherapy device 10 for one hour. They then filled out a survey for each phototherapy device 10. The questions focused on the user experience, determining if the light source 14 was too bright or distracting, if the user felt embarrassed to use it in public, if other people in close proximity to the user were distracted by the light, and other questions.

It was discovered that the optimal position of the light is directly in front of the user. This is contrary to what doctors and professionals prescribe for phototherapy treatment. Professionals suggest users set their lamp 45 degrees to their right or left because the large surface area of bright light is distracting and strains the eyes when aimed directly at them. However, our testing revealed that to have a visible increase in luminous flux through the eye, the light must be directly in front of the user. With the phototherapy device 10 set directly in front of the user on the top of their laptop, more light reaches the eye and therefore the treatment is more efficient.

It was also discovered that the intensity of light varies with the inverse square law, so as the distance increases from the light, the intensity of the light at that point decreases. The inverse square law is given by luminous flux through the eye=K/(Distance from the eye)̂2. This analysis was useful in determining how far away the user can be sitting from the light and still receive positive effects from the light.

Because portability is essential to users, the effect of the phototherapy device 10 on the battery life of computers was also tested. To test how the phototherapy device 10 affected a computer's battery, control tests were conducted leaving a fully charged laptop open with nothing plugged in and no applications open. The change in battery power levels were measured over the course of an hour, recording the number of battery life percentage decrease every ten minutes. Next, the phototherapy device 10 was plugged in to the computer, with the brightness on high, and the power consumption recorded every ten minutes for an hour. This test was to determine if various embodiments of the phototherapy device 10 greatly affected the battery life of a computer.

Spectral analysis ensured that the phototherapy device 10 emits the correct wavelengths of light clinically proven to improve SAD symptoms. There is some research that suggests wavelengths in the 460-490 nm range can cause photochemical damage to the eyes and those wavelengths should be avoided. The battery life tests proved that the phototherapy device 10 does not significantly increase the rate at which the battery power is depleted. Because the slope of the battery consumption did not change after plugging the phototherapy device 10 into the computer, the user will not have to change their charging habits for their computer when using the phototherapy device 10.

This invention has been described with reference to several preferred embodiments. Many modifications and alterations will occur to others upon reading and understanding the preceding specification. It is intended that the invention be construed as including all such alterations and modifications in so far as they come within the scope of the appended claims or the equivalents of these claims.

What is claimed is:

1. A phototherapy device for treatment of seasonal affective disorder that comprises:
a housing comprising:
a light source;
a mounting device that allows the phototherapy device to be mounted to any object or surface to allow said light source to be directed at a user's eye;
intensity control for modulating the intensity of light emitted by said light source; and
a power source to supply power to the phototherapy device;
the housing having a concave surface onto which said light source is mounted; and
the concave surface imparting a degree of curvature to said light source to enhance the amount of light directed towards the eye of a user adjusted for the typical distance of said light source from the user.

2. The phototherapy device of claim 1 further comprising a lens positioned to obscure the individual elements of said light source and to diffuse the light from said light source while maintaining the brightness of said light source.

3. The phototherapy device of claim 1 in which said mounting device allows the phototherapy device to be mounted to a laptop computer, a desktop monitor, a television screen, a handheld electronic device, a clipboard, or a table.

4. The phototherapy device of claim 1 in which said mounting device is any one of a clamp, an adhesive, a screw, an elastic band, a tripod, hook and loop fastening strips, or a stand.

5. The phototherapy device of claim 1 in which said power source is any one of a USB connection, a micro-USB connection, batteries, or a wired connection to a power outlet.

6. The phototherapy device of claim 1 in which said light source provides light in any of full spectrum visible spectrum, polychromatic white light, blue light, or a tunable spectrum.

7. The phototherapy device of claim 1 in which said light source is any one of LED lights, incandescent bulbs, fluorescent bulbs, or compact fluorescent bulbs.

8. The phototherapy device of claim 1 in which said intensity control is any one of a potentiometer or a power modulator.
